(19) United States
(12) Patent Application Publication
(10) Pub. No.: US 2025/0052764 A1
(43) Pub. Date: Feb. 13, 2025

(54) METHOD FOR PREDICTION OF FUTURE CARDIOVASCULAR DISEASE RISK VIA ANALYSIS OF IgG GLYCOME

(71) Applicants: GENOS d.o.o., Osijek (HR); The Brigham and Women’s Hospital, Inc., Boston, MA (US)

(72) Inventors: Branimir PLAVSA, Zagreb (HR); Gordan LAUC, Zagreb (HR); Samia MORA, Cambridge, MA (US); Rosangela AKEMI HOSHI, Miami, FL (US); Olga DEMLER, Boston, MA (US)

(73) Assignees: GENOS d.o.o., Osijek (HR); The Brigham and Women’s Hospital, Inc., Boston, MA (US)

(21) Appl. No.: 18/231,793

(22) Filed: Aug. 9, 2023

(51) Int. Cl. G01N 33/68

(52) U.S. Cl. CPC G01N 33/6854; G01N 33/6848; G01N 2800/32; G01N 2800/50

(57) ABSTRACT

The present disclosure reveals a process for the determination of a risk for the development of cardiovascular disease (CVD) in the future from quantitative analysis of four specific IgG N-glycans isolated from a blood sample of examined human subjects. Said four specific glycans were found to be significant diagnostic markers by statistical analysis of the results obtained from previously performed clinical trials that revealed the variation of quantitative IgG glycans content in the blood plasma in a cohort of normal subjects, versus, those who experienced CVD clinical manifestations. From this analysis, a new numerical model was developed which enables the calculation of the CVD risk parameter (CVDR). The numerical value for CVDR obtained after the inclusion of the result of said four key glycans, obtained from the blood analysis, gives an assessment of the eventual CVD development in the examined subject in the future.

### 1. TECHNICAL FIELD

The present disclosure relates to a method of predicting future cardiovascular disease (CVD) risk for a currently healthy person or persons. In particular, the present disclosure discloses a process for the determination of future cardiovascular disease (CVD) risk, based on quantitative analysis of N-glycans bound to immunoglobulin G (IgG) from a sample of the blood from examined subject. Cardiovascular disease (CVD), as used hereby, is any adverse cardiovascular event, e.g., myocardial infarction, stroke, or unstable angina.

### 2. BACKGROUND

Glycans are complex carbohydrates composed of different monosaccharides, primarily N-acetyl-glucosamine (▪), fucose (▾), mannose (●), galactose (∘), and sialic acid (♦). Glycans are covalently bound to proteins, usually via N-glycoside bond and are involved in a multitude of physiological and pathological processes. Due to their influence on a large number of biological processes, they are recognized as important biochemical markers of overall health as well as various physiological and pathological conditions of the human body, see literature reference 1:
1) G. Opdenakker, P. M. Rudd, C. P. Ponting, R. A. Dwek: Concepts and principles of glycobiology, FASEB J. 7 (1993) 1330-1337.

Immunoglobulin G (IgG) is the most prevalent antibody in human plasma, playing an important role in defending the body against various pathogens. IgG is a glycoprotein, and the glycans attached to its heavy chains are particularly important for its stability and function.

The glycosylation of IgG is dependent on various physiological (age, sex, pregnancy) and pathological conditions (tumors, infections, autoimmune diseases). Changes in the pattern of IgG glycosylation during aging are known in the art, and by monitoring IgG N-glycans, it is possible to derive conclusions about the biological age of the subject being studied, see literature references 2-5:
2) R. Parekh, I. Roitt, D. Isenberg, R. Dwek, T. Rademacher: Age-related galactosylation of the N-linked oligosaccharides of human serum IgG, J. Exp. Med. 167 (1988) 1731-1736,
3) M. Pučić, A. Knežević, J. Vidič, B. Adamczyk, M. Novokmet, O. Polašek, O. Gornik, S. Šupraha-Goreta, M. R. Wormald, I. Redžić, H. Campbell, A. Wright, N. D. Hastie, J. F. Wilson, I. Rudan, M. Wuhrer, P. M. Rudd, D. Josić, G. Lauc: High Throughput Isolation and Glycosylation Analysis of IgG-Variability and Heritability of the IgG Glycome in Three Isolated Human Populations, Mol. Cell. Proteomics 10.10; doi:10.1074/mcp.M111.010090,
4) EP3011335B1; G. Lauc, M. Pučić-Baković, F. Vučković: Method for the analysis of N-glycans attached to immunoglobulin G from human blood plasma and its use; applicant: Genos d.o.o. (HR); priority date: 20.06.2013, and
5) J. Krištić, F. Vučković, C. Menni, L. Klarić, T. Keser, I. Beceheli, M. Pučić-Baković, M. Novokmet, M. Mangino, K. Thaqi, P. Rudan, N. Novokmet, J. Sarac, S. Missoni, I. Kolčić, O. Polašek, I. Rudan, H. Campbell, C. Hayward, Y. Aulchenko, A. Valdes, J. F. Wilson, O. Gornik, D. Primorac, V. Zoldoš, T. Spector, G. Lauc: Glycans are a novel biomarker of chronological and biological ages, J. Gerontol. A Biol. Sci. Med. Sci. 69 (2014) 779-789. doi: 10.1093/gerona/glt190. The value of posttranslational modifications of proteins on cardiovascular disease (CVD) has received increasing attention due to the growing body of evidence showing their involvement in other human diseases, such as chronic inflammation, cancers, diabetes, and hypertension, see literature references 6 and 7:
6) I. Gudelj, G. Lauc: Protein N-Glycosylation in Cardiovascular Disease and Related Risk Factors, Curr. Cardiovasc. Risk Rep. (2018) 12, and
7) A. Birukov, B. Plavša, F. Eichelmann, O. Kuxhaus, R. A. Hoshi, N. Rudman, T. Štambuk, I. Trbojević-Akmačić, C. Schiborn, J. Morze, M. Mihelčić, A. Cindrić, Y. Liu, O. Demler, M. Perola, S. Mora, M. B. Schulze, G. Lauc, C. Wittenbecher: Immunoglobulin G N-Glycosylation Signatures in Incident Type 2 Diabetes and Cardiovascular Disease, Diabetes Care 45 (2022) 2729-2736.

Understanding how differences in the IgG N-glycosylation profile and the effects on IgG function relate to CVD events may lead to novel approaches to CVD prevention that leverages glycobiological mechanisms and pathways.

Glycans have an important role in atherosclerosis which is a chronic progressive inflammatory disease of the artery walls, see literature references 8 and 9:
8) A. O. Akinkuolie, J. E. Buring, P. M. Ridker, S. Mora: A novel protein glycan biomarker and future cardiovascular disease events, J. Am. Heart Assoc. 3 (2014) e001221, and
9) V. Eckardt, C. Weber, P. von Hundelshausen: Glycans and Glycan-Binding Proteins in Atherosclerosis, Thromb. Haemost. 119 (2019) 1265-1273. At the same time, alterations in protein glycosylation patterns can modulate IgG receptor binding, directly impacting inflammatory properties.

Since inflammation is both a cause and an aggravating factor in cardiometabolic and CVD, as well as a mediator of a worse prognosis, certain IgG glycosylation profiles may confer an at-risk phenotype for the development of CVD, see literature references 8, 10-12:
10) R. Dubé, G. A. W. Rook, J. Steele, R. Brealey, R. Dwek, T. Rademacher, J. Lennard-Jones: Agalactosyl IgG in inflammatory bowel disease: Correlation with C-reactive protein, Gut 31 (1990) 431-434,
11) O. M. Lardinois, L. J. Deterding, J. J. Hess, C. J. Poulton, C. D. Henderson, J. C. Jannette, P. H. Nachman, R. J. Falk: Immunoglobulins G from patients with ANCA-associated vasculitis are atypically glycosylated in both the Fc and Fab regions and the relation to disease activity, PLoS One 14 (2022) e0213215, and
12) C. Wittenbecher, T. Štambuk, 0. C. Kuxhaus, N. Rudman, F. Vučković, J. Štambuk, C. Schiborn, D. Rahelić, S. Dietrich, O. Gornik, M. Perola, H. Boeing, M. B. Schulze, G. Lauc: Plasma N-Glycans as Emerging Biomarkers of Cardiometabolic Risk: A Prospective Investigation in the EPIC-Potsdam Cohort Study, Diabetes Care 43 (2020) 661-668.

Literature reference 12 is focused on the analysis of total blood plasma glycans, where it was not known to which protein each glycan was bound. In the case of the present disclosure, the quantitative analysis is precisely targeted only to IgG glycans.

Menni and coworkers studied the correlation of glycans and one CVD risk index in two independent cohorts where it was found that IgG glycosylation traits are independently associated with subclinical atherosclerosis, while one specific trait related to the sialylated N-glycan is negatively correlated with CVD risk, very-low-density lipoprotein, and triglyceride serum levels, and presence of carotid plaque, see literature reference 13:
13) C. Menni, I. Gudelj, E. MacDonald-Dunlop, M. Mangino, J. Zierer, E. Bešić, P. K. Joshi, I. Trbojević-Akmačić, P. J. Chowienczyk, T. D. Spector, J. F. Wilson, G. Lauc, A. M. Valdes: Glycosylation Profile of Immunoglobulin G Is Cross-Sectionally Associated with Cardiovascular Disease Risk Score and Subclinical Atherosclerosis in Two Independent Cohorts, Circ. Res. 122 (2018) 1555-1564.

In contrast to the knowledge disclosed in the literature reference 13, the present disclosure reveals some specific IgG glycans that are significantly more effective predictors of CVD events.

The present disclosure solves the technical problem of the assessment of CVD development risk in the future at currently healthy subjects. This technical problem is solved in the present disclosure by the use of quantitative analysis of four specific IgG N-glycans that proved to be specific markers for the detection of said future CVD development in the future from one or more blood analyses of the examined subject.

### SUMMARY

The present disclosure discloses a diagnostic method for the prediction of future cardiovascular disease (CVD) in human subjects by an analysis of N-glycans I, bound to immunoglobulin G (IgG) from human blood plasma,

where the following symbols in I are used to denote monomeric sugar units:
▪ N-acetylglucosamine
▾ fucose
● mannose
∘ galactose
♦ sialic acid
   where the letters a-d determine the type of glycoside bond of said N-glycans I:

$$\begin{matrix}a = β ( 1 - 4 ) \\ b = α ( 1 - 6 ) \\ c = α ( 1 - 3 ) \\ d = β ( 1 - 2 )\end{matrix}$$

where the said diagnostic process comprises the following steps:
a) isolation of plasma from one or more blood samples that have been collected from the subject under examination,
b) isolation of IgG from blood plasma,
c) the release of said glycans from IgG,
d) fluorescent derivatization with 2-aminobenzamide (2AB) and suitable reducing agent for reductive amination, preferably using the complex of picoline borane (2PB; BH₃·NC₅H₄-2-CH₃) or sodium cyanoborohydride (NaBH₃CN)

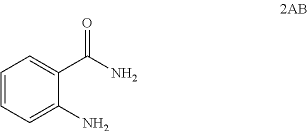

e) quantitative analysis of thus derivatized glycans by ultraperformance liquid chromatography (UPLC), wherein the numerical values of relative area under the respective glycan peaks GP1-GP24 within the corresponding UPLC chromatogram are obtained as the numerical values {GP1, GP2, . . . , GP24}, where the said glycan peaks GP1-GP24 belong to structures given in the table below:
f) wherein the numerical results of relative areas under the selected glycan peaks GP9, GP12, GP19, and GP20, that correspond to values {GP9, GP12, GP19, GP20}calculated in the previous step, are now included in the numerical model for cardiovascular diseases risk (CVDR), which is the function of the said four glycan arguments:

CVDR=CVDR(GP9,GP12,GP19,GP20)

and which estimates the risk value expressed as the CVDR value.

According to the present disclosure, the CVDR model is obtained as a result of statistical data analysis performed after a prospective clinical study that determines the variation of quantitative IgG glycans {GP1, . . . , GP24}content in the blood plasma in:
normal subjects, versus,
subjects who experienced the following cardiovascular disease clinical manifestations: myocardial infarction, stroke, coronary revascularization, unstable angina requiring hospitalization, or death,
   where the significantly predictive IgG glycans were determined using regression models that were corrected for multiple possible confounders, including age, race, sex, statin therapy, low-density lipoprotein cholesterol, high-density lipoprotein cholesterol, smoking, and hypertension. The determined IgG glycans were then used to construct a glycan-based predictive model.

The assessment CVDR method according to the present disclosure includes a numerical model as follows:

$$CVDR = - 3.432 \cdot GP 9 - 0.509 \cdot GP 12 + 0.793 \cdot GP 19 - 0.835 \cdot GP 20$$

The CVDR value gives a qualitative assessment of cardiovascular disease (CVD) onset in the future for the examined subject as follows:
(i) qualitatively, if the CVDR value is greater than the mean CVDR value of the population, the subject has an increased risk of cardiovascular events in the future compared to the reference population, and
(ii) quantitatively, the increase of CVDR value by 1 unit represents an increased risk of 2.6 fold.

The mean CVDR value of the population depends on the characteristics of a given population regarding the territorial area where the test would be applied and would have to be determined on a representative sample of a population.

In a further embodiment of this disclosure, the determination of the glycans under the peaks GP9, GP12, GP19, and GP20 can be performed by alternative quantitative analysis techniques selected from the group consisting of: MALDI-TOF mass spectrometry, liquid chromatography coupled with mass spectrometry (LC-MS), or capillary electrophoresis (CE), where the corresponding glycans can be derivatized with a fluorescent derivatizing agent selected from the group comprising 2-aminobenzamide (2AB), 8-aminopyrene-1,3,6-trisulfonic acid, trisodium salt (APTS), procainamide (PR), 2,5-dioxopyrrolidine-1-yl-[2N-(2-(N′,N′-diethylamino)ethyl)carbamoyl]-quinoline-6-yl-carbamate (RF), or other suitable derivatizing agents:

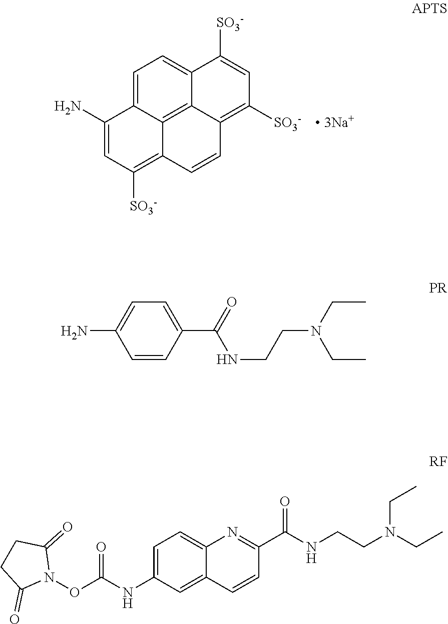

or by LC-MS analysis of the corresponding glycopeptides.
The diagnostic process according to the present disclosure is used for the determination of whether the examined subject has an increased risk of cardiovascular disease (CVD) development in the future.

### BRIEF DESCRIPTION OF DRAWINGS

In order to explain the technical features of embodiments of the present disclosure more clearly, the drawings used in the present disclosure are briefly introduced as follows. Obviously, the drawings in the following description are some exemplary embodiments of the present disclosure. Ordinary person skilled in the art may obtain other drawings and features based on these disclosed drawings without inventive efforts.

FIG. 1. Typical chromatogram of 2-amino-benzamide (2AB) derived IgG N-glycans obtained by the ultra-high performance liquid chromatography (HILIC-UPLC) described in Example 1, with 24 separated chromatographic peaks which are further in the text designed as GP1-GP24.

FIG. 2. HR and 95% CI for CVD events per SD increase in the relative abundance of IgG-GP1-24. Significant results for model 2 in JUPITER, adjusted for age, statin therapy, race, LDL-C, HDL-C, smoking, and hypertension (sex was a criterion for CVD case-control matching) and validated in TNT (the same covariates as in JUPITER plus sex and diabetes) are highlighted with marks ▪ and ▴, respectively. Error bars are 95% CI. Analyses were adjusted for multiple comparisons at the two-stage overall FDR level of 0.05 (FDR<0.2 in each stage).

FIG. 3. HR and 95% CI for CVD events per SD increase derived IgG N-glycosylation traits. Significant results for model 2 in JUPITER, adjusted for age, statin therapy, race, LDL-C, HDL-C, smoking, and hypertension (sex was a criterion for CVD case-control matching) and validated in TNT (the same covariates as in JUPITER plus sex and diabetes) are highlighted with marks ▪ and ▴, respectively. Error bars are 95% CI. Analyses were adjusted for multiple comparisons at the two-stage overall FDR level of 0.05 (FDR<0.2 in each stage). The composition of each particular IgG N-glycosylation trait B, CF, G0, G1, G2, S0, S1, and S2 are presented in Table 4.

FIG. 4. A flowchart for IgG-GP1-24 selection in JUPITER to compose the IgG glycan score. * Adjusted for age, statin therapy, race, LDL-C, HDL-C, smoking, and hypertension. ** The IgG glycan score, herein defined as CVDR was a linear combination of these 4 IgG GPs using the β-coefficients from the adjusted model controlled for age and sex, according to the present disclosure defined as:

$$CVDR = - 3.432 \cdot GP 9 - 0.509 \cdot GP 12 + 0.793 \cdot GP 19 - 0.835 \cdot GP 20$$

### DETAILED DESCRIPTION

The present disclosure reveals a diagnostic method for the prediction of future cardiovascular disease (CVD) in human subjects by an analysis of N-glycans I, bound to immunoglobulin G (IgG) from human blood plasma,

where the following symbols in I are used to denote monomeric sugar units:
▪ N-acetylglucosamine
▾ fucose
● mannose
∘ galactose
♦ sialic acid
   where the letters a-d determine the type of glycoside bond of said N-glycans I:

$$\begin{matrix}a = β ( 1 - 4 ) \\ b = α ( 1 - 6 ) \\ c = α ( 1 - 3 ) \\ d = β ( 1 - 2 )\end{matrix}$$

where the said diagnostic process comprises the following steps:
a) isolation of plasma from one or more blood samples that have been collected from the subject under examination,
b) isolation of IgG from blood plasma,
c) the release of said glycans from IgG,
d) fluorescent derivatization with 2-aminobenzamide (2AB) and suitable reducing agent for reductive amination, preferably using the complex of picoline borane (2PB; BH₃·NC₅H₄-2-CH₃) or sodium cyanoborohydride (NaBH₃CN):

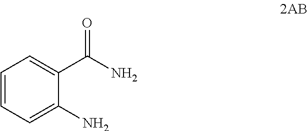

e) quantitative analysis of thus derivatized glycans by ultraperformance liquid chromatography (UPLC), wherein the numerical values of relative area under the respective glycan peaks GP1-GP24 within the corresponding UPLC chromatogram are obtained as the numerical values {GP1, GP2, . . . , GP24}, where the said glycan peaks GP1-GP24 belong to structures given in the table below:
f) wherein the numerical results of relative areas under the selected glycan peaks GP9, G212, 0219, and GP20, that correspond to values {GP9, GP12, GP19, GP20} calculated in the previous step, are now included in the numerical model for cardiovascular diseases risk: (CVDR), which is the function of the said four glycan arguments:

CVDR=CVDR(GP9,G212,G219,GP20)

and which estimates the risk: value expressed as the CVDR value.

According to the present disclosure, the CVDR model is obtained as a result of statistical data analysis performed after a prospective clinical trial study that determines the variation of quantitative IgG glycans {GP1, . . . , GP24} content in the blood plasma in:
normal subjects, versus,
subjects who experienced the following cardiovascular disease clinical manifestations: myocardial infarction, stroke, coronary revascularization, unstable angina requiring hospitalization, or death,
   where the significantly predictive IgG glycans were determined using regression models that were corrected for multiple possible confounders, including age, race, sex, statin therapy, low-density lipoprotein cholesterol, high-density lipoprotein cholesterol, smoking, and hypertension. The determined IgG glycans were then used to construct a glycan-based predictive model.

The assessment CVDR method according to the present disclosure includes a numerical model as follows:

$$CVDR = - 3.432 \cdot GP 9 - 0.509 \cdot GP 12 + 0.793 \cdot GP 19 - 0.835 \cdot GP 20$$

where said CVDR value gives a qualitative assessment of cardiovascular disease (CVD) onset in the future for the examined subject as follows:
(i) qualitatively, if the CVDR value is greater than the mean CVDR value of the population, the subject has an increased risk of cardiovascular events in the future compared to the reference population, and
(ii) quantitatively, the increase of CVDR value by 1 unit represents an increased risk of 2.6 fold,
   where the mean CVDR value of the population depends on the characteristics of a given population regarding the territorial area where the test would be applied and would have to be determined on a representative sample of a population.

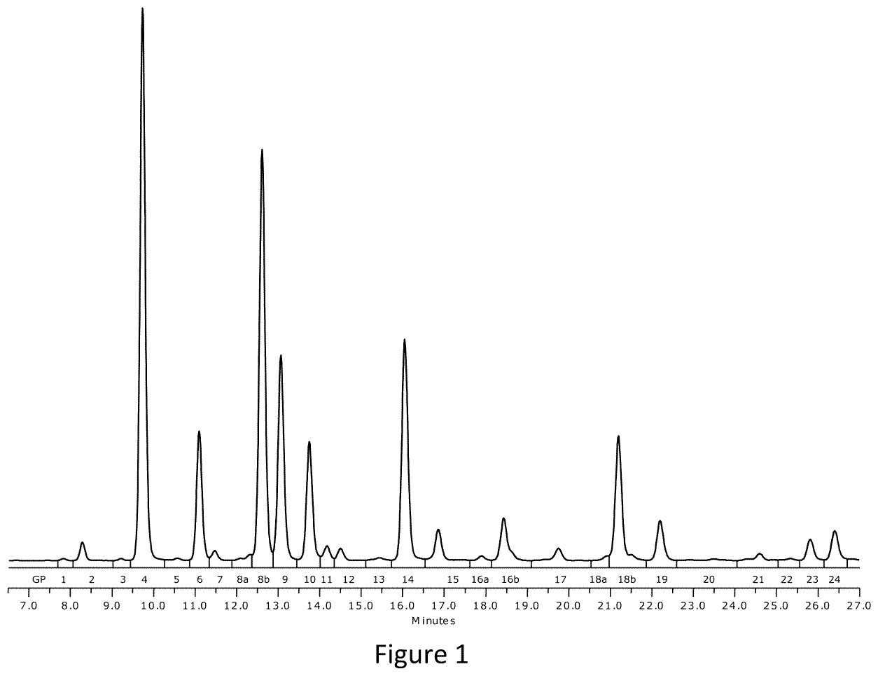

The quantitative determination of IgG N-glycans and particularly the key four glycan peaks GP9, GP12, GP19, and GP20 from the blood plasma of an examined subject is performed by the procedure described in Example 1. The typical chromatogram obtained by this method is presented in FIG. 1, while the retention times (RT) and glucose units (GU) of thus separated IgG glycans GP1-GP24 are given in Table 1.

TABLE 1
Retention times (RT) and glucose units (GU) for respective glycan
peaks GP1-GP24 as obtained by the UPLC analytical method described in
Example 1. The corresponding UPLC chromatogram is shown in FIG. 1.

| | Glycan | RT^{a} | |
|---|---|---|---|
| No. | peak | [min] | GU^{b} |
| 1 | GP1 | 7.8 | 5.45 |
| 2 | GP2 | 8.3 | 5.58 |
| 3 | GP3 | 9.2 | 5.84 |
| 4 | GP4 | 9.7 | 5.97 |
| 5 | GP5 | 10.6 | 6.17 |
| 6 | GP6 | 11.1 | 6.30 |
| 7 | GP7 | 11.5 | 6.38 |
| 8 | GP8 | 12.6 | 6.66 |
| 9 | GP9 | 13.1 | 6.77 |
| 10 | GP10 | 13.8 | 6.94 |
| 11 | GP11 | 14.2 | 7.04 |
| 12 | GP12 | 14.5 | 7.13 |
| 13 | GP13 | 15.4 | 7.38 |
| 14 | GP14 | 16.1 | 7.55 |
| 15 | GP15 | 16.9 | 7.78 |
| 16 | GP16 | 18.4 | 8.23 |
| 17 | GP17 | 19.8 | 8.61 |
| 18 | GP18 | 21.2 | 9.01 |
| 19 | GP19 | 22.2 | 9.28 |
| 20 | GP20 | 23.5 | 9.60 |
| 21 | GP21 | 24.6 | 9.88 |
| 22 | GP22 | 25.3 | 10.07 |
| 23 | GP23 | 25.8 | 10.20 |
| 24 | GP24 | 26.4 | 10.38 |

^{a}RT = retention time of the respective glycan peak.
^{b}GU (glucose units) represent the retention time calibrated to the retention times of glucose oligomers labeled with 2-aminobenzamide. This reduces the variation in retention times of the system between separate runs.

The study of IgG N-glycans in a large population of healthy subjects and subjects who developed CVD and the development of the numerical model for the calculation of CVDR according to the present disclosure is described in Example 2.

The procedure for performing the CVDR assessment method is described in Example 3.

In a further embodiment of this disclosure, the determination of the glycans under the peaks GP9, GP12, GP19, and GP20 can be performed by alternative quantitative analysis techniques selected from the group consisting of: MALDI-TOF mass spectrometry, liquid chromatography coupled with mass spectrometry (LC-MS), or capillary electrophoresis (CE), where the corresponding glycans can be derivatized with a fluorescent derivatizing agents selected from the group comprising 2-aminobenzamide (2AB), 8-aminopyrene-1,3,6-trisulfonic acid, trisodium salt (APTS), procainamide (PR), 2,5-dioxopyrrolidine-1-yl-[2N-(2-(N′,N′-diethylamino)ethyl)carbamoyl]-quinoline-6-yl-carbamate (RF), or other suitable derivatizing agents:

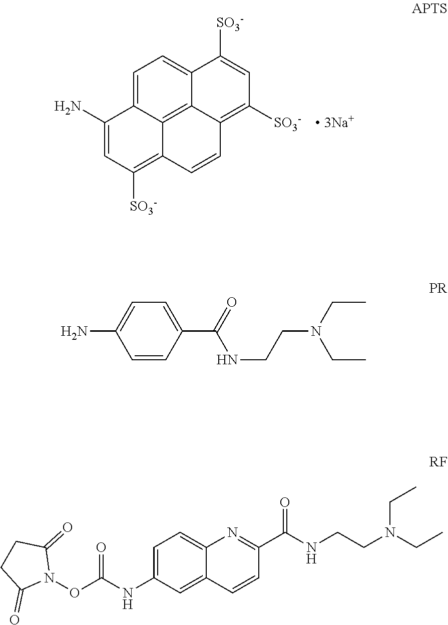

or by LC-MS analysis of the corresponding glycopeptides.

### Use of the Diagnostic Process According to the Present Disclosure

The diagnostic process according to the present disclosure is used for the determination of whether the examined subject has an increased risk of cardiovascular disease (CVD) development in the future.

If the human subject is determined to have an increased risk of cardiovascular events in the future, suitable compounds or treatments could be administered to the examined subject to reduce or prevent the risk. Suitable compounds may include, but are not limited to low-dose acetylsalicylic acid, quercetin, resveratrol, N-acetyl-D-mannosamine, or a combination of them. For example, in a reference, Peng, J., et al. (2019), Supplementation with the Sialic Acid Precursor N-acetyl-D-Mannosamine Breaks the Link Between Obesity and Hypertension. Circulation. doi:10.1161/circulationaha.119.043490, which is incorporated herein by reference in its entirety, assays show that administering of the said substance, i.e., N-acetyl-D-mannosamine, repairs the mice's IgG glycome as well as prevents the onset of hypertension as a consequence of obesity.

### Experimental Part

### General Information

Chemicals, reagents, and accessories used in this research are purchased from the following suppliers: 2-aminobenzamide (2AB): Sigma-Aldrich (US); 2-picoline borane (2PB): Sigma-Aldrich (US); acetonitrile, HPLC grade: Scharlab; acetonitrile LC-MS grade: J. T. Baker (US); ammonium chloride (NH₄Cl): Acros Organics (BE); dimethyl sulfoxide (DMSO): Sigma-Aldrich (US); ethanol: Carlo Erba (IT); formic acid (HCOOH): Merck (DE); Igepal CA-630: Sigma-Aldrich (US); potassium dihydrogen phosphate (KH₂PO₄): Sigma-Aldrich (US); potassium chloride (KCl): EMD Millipore (US); hydrochloric acid (HCl): Kemika (HR); sodium dodecylsulfate (SDS): Sigma-Aldrich (US); sodium hydrogen phosphate (Na₂HPO₄): Acros Organics (BE); sodium hydrogen carbonate (NaHCO₃): Merck (DE); sodium hydroxide (NaOH): Kemika (HR); sodium chloride (NaCl): Carlo Erba (IT); acetic acid (CH₃COOH): Merck (DE); ammonia solution: Merck (DE); tris(hydroxymethyl)aminometane: Acros Organics (BE); ultrapure water: Millipore (US); PNGase F (10 U/mL): Promega; ARC 2^{nd} Gen Testo RGT: Abbott Diagnostics (US); ARC Trigger solution: Abbott Diagnostics (US); ARC Pre-trigger solution: Abbott Diagnostics (US); GHP Acroprep 0.20 μm filter plate: Pall Corp. (US); GHP Acroprep 0.45 μm filter plate: Pall Corp. (US); Supor PES filter: Nalgene (US); plate for sample collection with 96-wells, 1-2 mL volume: Waters (US); Protein G monolithic plate (96-wells): Sartorious BIA Separations (SI).

The research was performed by the use of the following instruments: ARCHITECT® i1000SR analyzer: Abbott Diagnostics (US); ABgene PCR plates: Thermo Scientific (US); Acquity UPLC Glycan BEH amide column, 130 Å, 1.7 μm, 2.1 mm×100 mm: Waters (US); Acquity UPLC H-Class system: Waters (US); reaction tubes ARC: Abbott Diagnostics (US); centrifuge, model 5840: Eppendorf (DE); Fume cupboard DIGIM 15 AFM: Schneider (FR); Water purification system Direct-Q 3UV: Millipore (US); analytical balance Explorer®: Ohaus Corporation (US); pH-meter FiveEasy™: Mettler Toledo (CH); precise balance JL1502-G: Mettler Toledo (CH); laboratory oven LAB. HOT AIR OVEN, M.R.C.; laboratory incubator: M.R.C.; centrifuge miniSpin: Eppendorf (DE); magnetic stirrer MR 3000 D: Heildoph (DE); spectrophotometer Nanodrop ND-8000: Thermo Scientific (US); Pipet-Lite XLS manual micropipette Rainin: Mettler Toledo (CH); circular shaker, model 3023: GFL; vacuum concentrator Savant SC210A SpeedVac and Savant solvent trap: Thermo Scientific (US); Refrigerated Vapor Traps RVT400 and vacuum pump OFP400: Thermo Scientific (US); vacuum manifold and vacuum pump: Pall (US); laboratory shaker Vortex-Genie 2: Scientific Industries (US).

The isolations of blood plasma samples from female subjects were performed by the methodology known in the prior art, see literature reference 4.

### Example 1. Isolation of Immunoglobulin G (IgG) from Human Plasma, Deglycosylation of IgG, Glycans Purification, Fluorescent Derivatization of Glycans with 2-Aminobenzamide (2AB) and Quantitative Analysis of Thus Released and Labeled Glycans

The isolation of IgG from blood plasma was conducted by the common process known in the prior art, see literature references 3 and 14:
14) I. Trbojević Akmačić, I. Ugrina, G. Lauc: Comparative Analysis and Validation of Different Steps in Glycomics Studies, Methods Enzymol. 586 (2017) 37-55. doi: 10.1016/bs.mie.2016.09.027.

### Isolation of IgG

IgG was isolated from the blood plasma samples by affinity chromatography by binding to a 96-wells protein G plate with a vacuum device for the plate filtration. All steps of IgG isolation were carried out at 380 mmHg pressure, while at the application of plasma samples and IgG elution, the reduced pressure at around 200 mmHg was employed. The solutions used for the isolation were previously filtered through a 0.2 μm filter (Supor PES filter). Before the application of the plasma samples, the protein G plate was washed with 2 mL ultrapure water (18 MΩ/cm at 25° C.), 2 mL concentrated PBS buffer, pH=7.4 (137 mmol/L NaCl, 2.7 mmol/L Na₂HPO₄, 9.7 mmol/L KH₂PO₄, 2.2 mmol/L KCl; titrated with NaOH to pH=7.4), 1 mL 0.1 mol/L formic acid, pH=2.5; 2 mL 10× concentrated PBS buffer, pH=6.6; and adjusted with 4 mL 1× concentrated PBS buffer, pH=7.4. Plasma samples of subjects (100 μL) and five aliquots of standard plasma samples (50 μL) were randomly added to each plate, while one well was left empty as a negative control. The plasma samples were mixed and centrifuged at 1,479 g for 10 minutes. Then, the samples were diluted by the addition of 1× concentrated PBS buffer, pH=7.4 in ratio 1:7, V/V, and filtered through 0.45 μm GHP AcroPrep filter plate with 96 wells, by the use of a vacuum device for plates (Pall). Filtered plasma samples were applied on a protein G plate and washed 3× 2 mL 1× concentrated PBS buffer, pH=7.4 to remove unbounded proteins. The bounded IgG was eluted from the protein G plate with 1 mL 0.1 mol/L formic acid and neutralized with 170 μL 1 mol/L ammonium hydrogen carbonate. Protein G plate was regenerated for repeated use by washing with 1 mL 0.1 mol/L formic acid, 2 mL 10× concentrated PBS buffer, pH=6.6., 4 mL 1× concentrated PBS buffer, pH=7.4, and 1 mL buffer for storage of protein G plate (ethanol, σ=20%; 20 mmol/L tris; 0.1 mol/L NaCl; titrated with HCL up to pH=7.4) and additional 1 mL of the buffer for storage was added and stored at +4° C.

### Determination of IgG Concentration

The IgG concentration was determined by measuring absorbance at 280 nm with a Nanodrop ND-8000 spectrophotometer (Thermo Scientific; US). A part of IgG eluate was separated and dried in rotary vacuum concentrator SpeedVac Concentrator SC210A (Thermo Scientific; US). Thus prepared samples were stored at −20° C. till further use.

### The Denaturation and Deglycosylation of IgG in Solution

Dried IgG samples were denatured with 30 μL SDS (γ=1.33?) and incubated at 65° C. for 10 minutes. 10 μL of Igepal CA-630 solution (γ=4%) was added to each sample to deactivate the excess SDS. The plates are incubated at r.t. for 15 minutes. IgG molecules were deglycosylated by the addition of 10 μL 5× concentrated PBS buffer and 1.25 U PNGase F. The deglycosylation reaction was conducted at 37° C. for 18 h.

Fluorescent Labelling with 2-Amino-Benzamide (2AB) and Purification of 2AB-Derivatised IgG N-Glycans

Due to the fact that glycans do not contain chromophores, their content cannot be measured by any spectrophotometric technique. This is the reason why free N-glycans are derivatized with fluorescent reagents such as 2AB. The derivatization reaction was carried out with 2AB (γ=19.2 mg/mL) and 2-picoline borane (2PB; γ=44.8 mg/mL) dissolved in the solution of acetic acid (HOAc) and DMSO in a ratio of 30:70, V/V. To each sample, per 25 μL of the said labeling solution was added and the samples were incubated at 65° C. for 2 h. After the derivatization reaction, all impurities were removed by solid phase extraction (HILIC-SPE). After cooling to r.t. for 30 minutes, to each sample, 700 μL acetonitrile (φ=100%, 4° C.) was added and the samples were transferred to GHP AcroPrep 0.2 μm filter plate. The filter plate was previously washed with 200 μL ethanol (φ=70%), 200 μL ultrapure water, and cooled acetonitrile (φ=96%, 4° C.). Between each of these steps, the filter plate was emptied with a vacuum manifold and the vacuum was not higher than 2 inHg. After transferring to GHP AcroPrep 0.2 μm filter plate, the samples were incubated at r.t. for 2 minutes. During this period, the binding of 2AB-labelled N-glycans to polypropylene membrane plate occurred. Each sample was then washed 4× with 200 μL cooled acetonitrile (φ=96%, 4° C.) and then the glycans were eluted from the membrane plate. The elution was conducted in two subsequent equal steps: to each well, 90 μL of ultrapure water was added followed by incubation at r.t. for 15 minutes with shaking on a circular shaker. Collected eluates were centrifuged at 164 g for 5 minutes into ABgene PCR plates. Purified fluorescently labeled IgG N-glycans were stored at −20° C. till further use.

### Analysis of IgG Glycans by UPLC Method

Labeled IgG N-glycans were analyzed by the HILIC-UPLC method on amide ACQUITY UPLC® Glycan BEH column (Waters; US) of 100 mm length, diameter 2.1 mm and particles size 1.7 μm according to the method described in literature reference 3. The analyses were conducted on Acquity UPLC H-Class (Waters; US) instrument equipped with quaternary solvent manager QSM, sample manager SM) and fluorescent (FLR) detector. The instrument was controlled by the program Empower 3, version 3471 (Waters; US).

The glycan samples were prepared by mixing with acetonitrile (φ=100%) in the ratio: sample:acetonitrile=20:80, V/V. As the mobile phase, ammonium formate, c=0.1 mol/L, pH=4.4 was used as solvent A and acetonitrile (φ=100%) as solvent B. Between analyses, the system was washed with aqueous acetonitrile (φ=75%). Samples were cooled to 10° C. before injecting, while the separation was carried out at 60° C. The analytical method uses a linear gradient with 25-38% solvent A at a flow rate of 0.4 ml/min, with 29 minutes run. Separated glycans were detected by FLR detector at a wavelength for 2AB: λₑₓ=250 nm, λₑₘ=428 nm). The system was calibrated with fluorescently labeled glucose oligomers as an external standard.

The typical chromatogram obtained by this method is presented in FIG. 1, while the retention times (RT) and glucose units (GU) of thus separated IgG glycans GP1-GP24 are given in Table 1.

### Example 2. The Study of IgG N-Glycans in a Large Population of Healthy Subjects and Subjects Who Developed CVD and the Development of the Numerical Model for the Calculation of CVDR According to the Present Disclosure

### Study Populations

The present study involved a nested case-control (matched for age and sex) of incident CVD cases from the Justification for the Use of Statins in Prevention: An Intervention Trial Evaluating Rosuvastatin (JUPITER; ClinicalTrials.gov: NCT00239681) as the discovery primary prevention cohort (N=162 case-control pairs), see literature reference 15:
15) P. M. Ridker, E. Danielson, F. A. H. Fonseca, J. Genest, A. M. Gotto, J. J. P. Kastelein, W. Koenig, P. Libby, A. J. Lorenzatti, J. G. MacFadyen, B. G. Nordestgaard, J. Shepherd, J. T. Willerson, R. J. Glynn (JUPITER Study Group): Rosuvastatin to Prevent Vascular Events in Men and Women with Elevated C-Reactive Protein, N. Eng. J. Med. 359 (2008) 2195-2207.

Briefly, JUPITER was a prospective (median follow-up of 1.9 years), randomized, double-blinded, and placebo-controlled trial for primary prevention in 17,802 participants with elevated high-sensitivity C-reactive protein (hs-CRP>2 mg/L) and average to low levels of low-density lipoprotein cholesterol (LDL-C<130 mg/dl) investigated for the effects of 20 mg/day rosuvastatin vs. placebo. Participants provided written informed consent at the time of enrollment. Institutional review board approval was obtained from Mass General Brigham (Boston, MA). The first and senior authors had full access to all data in the study and take responsibility for their integrity and data analysis.

Significant associations from JUPITER were then validated in a sub-study of the Treating to New Targets (TNT; ClinicalTrials.gov: NCT00327691) trial, a prospective (mean follow-up of 5.5 years), randomized, multicenter study comparing the efficacy of high-dose (80 mg) vs. usual-dose atorvastatin (10 mg) for the secondary prevention of cardiovascular events in 10,001 randomized patients with clinically evident coronary heart disease, see literature reference 16:
16) J. C. LaRosa, S. M. Grundy, D. D. Waters, C. Shear, P. Barter, J. -C. Fruchart, A. M. Gotto, H. Greten, J. J. P. Kastelein, J. Shepherd, N. K. Wenger: Intensive Lipid Lowering with Atorvastatin in Patients with Stable Coronary Disease, N. Engl. J. Med. 352 (2005) 1425-1435.

Three-hundred and sixty-seven CVD nested case-control pairs of participants from the main trial were matched for low- or high-dose statin therapy and a disease risk score comprised of 17 cardiovascular clinical and biomarker risk factors, see literature reference 17:
17) S. Mora, N. K. Wenger, D. A. Demicco, A. Breazna, S. M. Boekholdt, B. J. Arsenault, P. Deedwania, J. J. P. Kastelein, D. D. Waters: Determinants of residual risk in secondary prevention patients treated with high-versus low-dose statin therapy: The treating to new targets (TNT) study, Circulation 125 (2012) 1979-1987.

All patients gave written informed consent, and the study was approved by the local research ethics committee or institutional review board at each center.

### IgG N-Glycan Profiling

Paired case-control plasma samples were placed in neighboring wells in random order throughout multi-well plates, and laboratory personnel was blinded to case status. IgG was isolated from individual plasma samples using 96-well protein G monolithic plates, eluted with 0.1 mol/L formic acid, and neutralized with 1 mol/L ammonium bicarbonate as previously described in detail, see literature reference 3. Prepared samples were stored at −20′C until ultraperformance liquid chromatography analysis on a Waters ACQUITY UPLC H-Class instrument. All chromatograms were separated in the same manner into 24 IgG glycan peaks GP1-GP24, and the amount of glycans in each peak was expressed as the percentage of the total integrated area, see Table 1 and FIG. 1.

In addition to 24 directly measured IgG glycans, 8 IgG N-glycosylation traits were derived: agalactosylation (G0), monogalactosylation (G1), digalactosylation (G2), asialylation (S0), monosialylation (S1), disialylation (S2), bisecting GlcNAc (B), and core fucosylation (CF). Derived traits were calculated as percentages of the sums of glycans with specific common structural features, see literature reference 7:

### Cardiovascular Disease Outcomes

JUPITER cases were defined as confirmed post-randomization myocardial infarction, stroke, coronary revascularization, unstable angina requiring hospitalization, or death. In TNT, CVD was defined as post-randomization nonfatal non-procedure-related myocardial infarction, resuscitation after cardiac arrest, fatal or nonfatal stroke, and coronary heart disease death. All events were adjudicated and confirmed by a medical review by the respective clinical trial endpoint committees, see literature references 15 and 16.

### Clinical and Biomarker Risk Factors

Baseline questionnaires were used to collect sex, age, race/ethnicity, body weight and height, use of non-randomized supplements or medications, smoking, and other relevant aspects of health history. In both studies, standard fasting lipid panels were obtained in a central laboratory as part of the clinical trial. LDL-C concentrations were calculated by the Friedewald equation when triglycerides were <400 mg/dL and measured by ultracentrifugation when ≥400 mg/dL, see literature references 18-19:
18) B. van den Bogaard, B. J. H. van den Born, R. Fayyad, D. D. Waters, D. A. DeMicco, J. C. LaRosa, J. J. P. Kastelein, I. Holme: On-treatment lipoprotein components and risk of cerebrovascular events in the Treating to New Targets study, Eur. J. Clin. Invest. 41 (2011) 134-142, and
19) S. Mora, R. J. Glynn, S. M. Boekholdt, B. G. Nordestgaard, J. J. P. Kastelein, P. M. Ridker: On-Treatment Non-High-Density Lipoprotein Cholesterol, Apolipoprotein B, Triglycerides, and Lipid Ratios in Relation to Residual Vascular Risk After Treatment with Potent Statin Therapy, J. Am. Coll. Cardiol. 59 (2012) 1521-1528.

In JUPITER, hs-CRP was measured using a high-sensitivity assay (Behring Nephelometer), see literature reference 19. In TNT, hs-CRP was measured at Quest Diagnostics (San Juan Capistrano, CA) using a nephelometric method with latex particles coated with CRP monoclonal antibodies. In addition, glycoprotein acetylation biomarker (GlycA), secretory phospholipase A2 (sPLA2), and lipoprotein-associated phospholipase A2 (LpPLA2) mass and activity were measured in JUPITER. GlycA signals were quantified at LipoScience Inc (Raleigh, NC) from plasma nuclear magnetic resonance spectra obtained from the automated NMR Profiler system, see literature reference 8.

sPLA2 was measured at Quest Diagnostics Nichols Institute (San Juan Capistrano, CA) with a commercially available enzyme immunoassay (Cayman assay; Cayman Chemical Co. Ann Arbor MI) based on a double-antibody sandwich technique that is specific for sPLA2-IIA, see literature reference 20:
20) A. O. Akinkuolie, P. R. Lawler, A. Y. Chu, M. Caulfield, J. Mu, B. Ding, F. Nyberg, R. J. Glynn, S. Mora: Group IIA Secretory Phospholipase A2 and Incident Cardiovascular Disease: An Analysis from the JUPITER Trial HHS Public Access, Arterioscler. Thromb. Vasc. Biol. 39 (2019) 1182-1190.

Concentrations of LpPLA2 mass were determined by a latex particle-enhanced turbidimetric immunoassay for LpPLA2 run on the Roche P-modular analyzer (PLAC™ test, diaDexus). LpPLA2 activity was measured in a research-use automated enzyme assay system, run on the Roche P-modular analyzer (CAM assay, diaDexus) with a colorimetric substrate that is converted upon hydrolysis by the phospholipase enzyme, see literature reference 21:
21) P. M. Ridker, J. G. McFadyen, R. L. Wolfert, W. Koenig: Relationship of Lipoprotein-Associated Phospholipase A2 Mass and Activity with Incident Vascular Events among Primary Prevention Patients Allocated to Placebo or to Statin Therapy: An Analysis from the JUPITER Trial, Clin. Chem. 58 (2012) 877-886.

### Statistical Analyses

IgG-GPs were globally normalized and log-transformed because of the right-skewness of their distributions. All measurements were adjusted for batch and run-day effects using ComBat (R-package sva³⁷), see literature reference 22:
22) J. T. Leek, W. E. Johnson, H. S. Parker, A. E. Jaffe, J. D. Storey: The SVA package for removing batch effects and other unwanted variation in high-throughput experiments, Bioinformatics 28 (2012) 882-883.

Outliers (< or >6 SD from the mean) were trimmed to mean±6SD, see literature reference 13. Derived glycan traits were calculated using normalized and batch-corrected glycan measurements (exponential of batch-corrected measurements). All variables were centered to mean=0 and scaled to SD=1 to allow for comparison of the effect estimates, see literature reference 7.

Risk associations with CVD of individual IgG N-glycans and derived IgG glycosylation traits were tested using conditional logistic regression models. In our discovery cohort, JUPITER, the minimally adjusted model 1 was fitted adjusting for age and randomized treatment assignment (statin therapy or placebo; cases and controls were matched on sex). Model 2 was adjusted for the same variables in model 1 plus race, LDL-C, HDL-C, hypertension, and current smoking. Model 3 was adjusted for the variables in model 2 plus body mass index (BMI), and hs-CRP. Using the same models, we exploratorily examined if there was effect modification by age (above and below the median) or sex.

In TNT, validation model 1 included age and sex (CVD cases and controls were matched on disease risk score and intervention arm [low vs. high statin dose]), see literature reference 17. Model 2 in TNT was adjusted for the same variables in model 1 plus race, LDL-C, HDL-C, hypertension, current smoking, and diabetes. Model 3 additionally included BMI and hs-CRP. Since we used risk set sampling for CVD case-control selection, the results were reported as hazard ratios (HR) and 95s confidence interval (95% CI).

All regressions were controlled for multiple testing using a two-stage procedure proposed by Benjamini and Yekutieli, setting FDR levels at 0.2 for each stage. Briefly, this approach guarantees an overall FDR correction <0.05 by the multiplication of the FDR levels from each stage, see literature reference 23:
23) Y. Benjamini, D. Yekutieli: The control of the false discovery rate in multiple testing under dependency, Ann. Statist. 29 (2001) 1165-1188.

Then, IgG-GPs significantly associated with CVD in JUPITER according to this criterion (FDR<0.2) were carried over for validation in TNT.

### IgG Glycan Score

We derived an IgG glycan score from a linear combination of IgG N-glycosylation traits associated with CVD risk in JUPITER. First, for variable selection and dimension reduction, we used the least absolute shrinkage and selection operator (LASSO) approach to fit a model that regresses the CVD outcome on all of the GPs simultaneously, applying a penalty on the magnitude of regression coefficients to achieve sparse variable selection (R-package clogitL1), see literature references 24 and 25:
24) R. Tibshirani: Regression Shrinkage and Selection via the Lasso, J. R. Statist. Soc. B 58 (1996) 267-288, and
25) S. Reid, R. Tibshirani: Regression: The clogitL1 Package, J. Statist. Soft. 58 (2014) 1-23.

GPs with non-zero LASSO regression coefficients were significantly selected in association with CVD. The optimal tuning parameter for the penalty term was selected via cross-validation. Then, still in JUPITER dataset, a conditional logistic regression for CVD was fitted with all selected GPs in a model adjusted for age, statin therapy, race, LDL-C, HDL-C, hypertension, and current smoking. Non-significant GPs were excluded, and significantly associated ones were included simultaneously in a newly adjusted model controlled for age and statin therapy. This last model generated the coefficients for the linear combination used for the IgG glycan score in both JUPITER and TNT subsets. The calculated glycan score was centered to mean=0 and scaled to SD=1.

We used a parametric approach to calculate the area under the curve (AUC) for the IgG glycan score in these two CVD case-control subsamples by adjusting the prediction models based on the parent studies of each trial. Then, we used the exact formula for AUC under the assumption of normality of predictor variables (normality was checked using the Shapiro-Wilk's test, and non-normal variables were transformed using Yeo-Johnson's Power Transformation [R-package VGAM]), see literature references 26 and 27:
26) I. N. K. Yeo, R. A. Johnson: A new family of power transformations to improve normality or symmetry, Biometrika 87 (2000) 954-959, and
27) T. W. Yee: The VGAM Package for Categorical Data Analysis, J. Stat. Softw. 32 (2010) 1-34.

To avoid over-optimism in AUC, the IgG glycan score in one population was calculated using the β-coefficients derived in the other. To obtain consistent estimates of 95% confidence intervals, we performed a bootstrap resampling procedure with 200 bootstrap replicates.

All statistical analyses were performed using the R software (v 4.1.0), see literature reference 28:
28) R Core Team. R: A Language and Environment for Statistical Computing (2019); available from: https://www.r-project.org/

### Participant Characteristics

Table 2 displays baseline characteristics of JUPITER (discovery cohort) and TNT (validation cohort) participants according to CVD case-control status. Groups in both sub-studies were well-balanced except for race, BMI, and smoking in JUPITER, and hs-CRP in TNT.

TABLE 2
Baseline characteristics of participants in the discovery primary prevention
cohort (JUPITER) and the validation secondary prevention cohort (INT).

| | JUPITER* | | | | | | |
|---|---|---|---|---|---|---|---|
| | Overall | | Controls | | Cases | | P- |
| Characteristic | (N = 324) | | (N = 162) | | (N = 162) | | value |
| Age (years) ‡ | 70.0 | [64.8, 75.0] | 70.0 | [64.3, 75.0] | 70.0 | [65.0, 75.8] | 0.947 |
| Women, N (%) | 78 | (24.1) | 39 | (24.1) | 39 | (24.1) | 1.000 |
| Race or ethnic | | | | | | | 0.026 |
| group | | | | | | | |
| White, N (%) | 273 | (84.3) | 143 | (88.3) | 130 | (80.2) | |
| African | 27 | (8.3) | 7 | (4.3) | 20 | (12.3) | |
| American, N (%) | | | | | | | |
| Other, N (%) | 24 | (7.4) | 12 | (7.4) | 12 | (7.4) | |
| BMI (kg/m²) ‡ | 27.60 | [24.9, 30.9] | 28.41 | [25.6, 31.3] | 26.94 | [23.9, 30.0] | 0.002 |
| Hypertension, | 190 | (58.6) | 97 | (59.9) | 93 | (57.4) | 0.735 |
| N (%) | | | | | | | |
| Current smoker, | 56 | (17.3) | 19 | (11.7) | 37 | (22.8) | 0.012 |
| N (%) | | | | | | | |
| HDL-C (mg/dL) ‡ | 48 | [40, 60] | 48 | [40, 60] | 49 | [41, 61] | 0.872 |
| LDL-C (mg/dL) ‡ | 110 | [94, 120] | 109 | [97, 120] | 111 | [92, 120] | 0.672 |
| Total-C (mg/dL) ‡ | 187 | [166, 201] | 188 | [166, 201] | 185 | [166, 200] | 0.284 |
| Triglycerides | 114 | [82, 166] | 114 | [81, 173] | 114 | [82, 165] | 0.538 |
| (mg/dL) ‡ | | | | | | | |
| hs-CRP (mg/L) ‡ | 4.40 | [2.8, 8.0] | 4.40 | [2.9, 7.7] | 4.35 | [2.8, 8.2] | 0.865 |
| Randomized statin | 136 | (42.0) | 67 | (41.4) | 69 | (42.6) | 0.910 |
| allocation, N (%) | | | | | | | |
| | TNT † | | | | | | |
| | Overall | | Controls | | Cases | | P- |
| Characteristic | (N = 734) | | (N = 367) | | (N = 367) | | value |
| Age (years) ‡ | 64.5 | [57.2, 70.0] | 63.8 | [56.9, 70.0] | 64.7 | [57.9, 69.7] | 0.545 |
| Women, N (%) | 123 | (16.8) | 53 | (14.4) | 70 | (19.1) | 0.114 |
| Race or ethnic | | | | | | | 0.916 |
| group | | | | | | | |
| White, N (%) | 673 | (91.7) | 337 | (91.8) | 336 | (91.6) | |
| Black, N (%) | 28 | (3.8) | 13 | (3.5) | 15 | (4.1) | |
| Other, N (%) | 33 | (4.5) | 17 | (4.6) | 16 | (4.4) | |
| BMI (kg/m²) ‡ | 28.4 | [25.8, 32.0] | 28.2 | [25.6, 31.2] | 28.6 | [25.9, 32.3] | 0.070 |
| Diabetes, N (%) | 168 | (22.9) | 86 | (23.4) | 82 | (22.3) | 0.792 |
| Hypertension, | 505 | (68.8) | 251 | (68.4) | 254 | (69.2) | 0.873 |
| N (%) | | | | | | | |
| Current smoker, | 123 | (16.8) | 59 | (16.1) | 64 | (17.4) | 0.693 |
| N (%) | | | | | | | |
| Disease risk | 0.62 | [0.2, 1.1] | 0.6 | [0.2, 1.1] | 0.6 | [0.2, 1.1] | 0.995 |
| score § | | | | | | | |
| HDL-C (mg/dL) ‡ | 44 | [38, 50] | 43 | [38, 50] | 44 | [38, 51] | 0.938 |
| LDL-C (mg/dl) ‡ | 98 | [87, 111] | 98 | [86, 111] | 98 | [88, 110] | 0.840 |
| Total-C (mg/dL) ‡ | 175 | [158, 191] | 176 | [158, 192] | 174 | [160, 190] | 0.754 |
| Triglycerides | 140 | [107, 199] | 140 | [108, 199] | 140 | [107, 200] | 0.692 |
| (mg/dL) ‡ | | | | | | | |
| hs-CRP (mg/L) ‡ | 0.80 | [0.30, 2.20] | 0.74 | [0.20, 2.14] | 0.90 | [0.40, 2.37] | 0.036 |
| Randomized | 338 | (46.0) | 169 | (46.0) | 169 | (46.0) | 1.000 |

high-statin
allocation, N (%)

*JUPITER case-control matching was for age and sex.
† TNT case-control matching was for high vs. low-intensity statin randomization and disease risk score.
‡ Median [IQR].
§ Calculated based on age, sex, smoking, hypertension, diabetes, BMI, myocardial infarction, angina, cerebrovascular disease, peripheral vascular disease, congestive heart failure, coronary revascularization, apolipoprotein B, use of calcium channel blockers, use of aspirin, and baseline blood urea nitrogen³⁰.
Abbreviations: BMI—body mass index; LDL-C—low-density lipoprotein cholesterol; HDL-C—high-density lipoprotein cholesterol; Total-C—total cholesterol; hs-CRP—high-sensitivity C-reactive protein.

Associations of Individual IgG-GPs with CVD

In JUPITER, FDR-adjusted significant differences were observed in IgG-GP9 and IgG-GP16 (cases<controls); in TNT, differences were detected in IgG-GP4 (cases>controls), IgG-GP13, and IgG-GP15 (cases<controls).

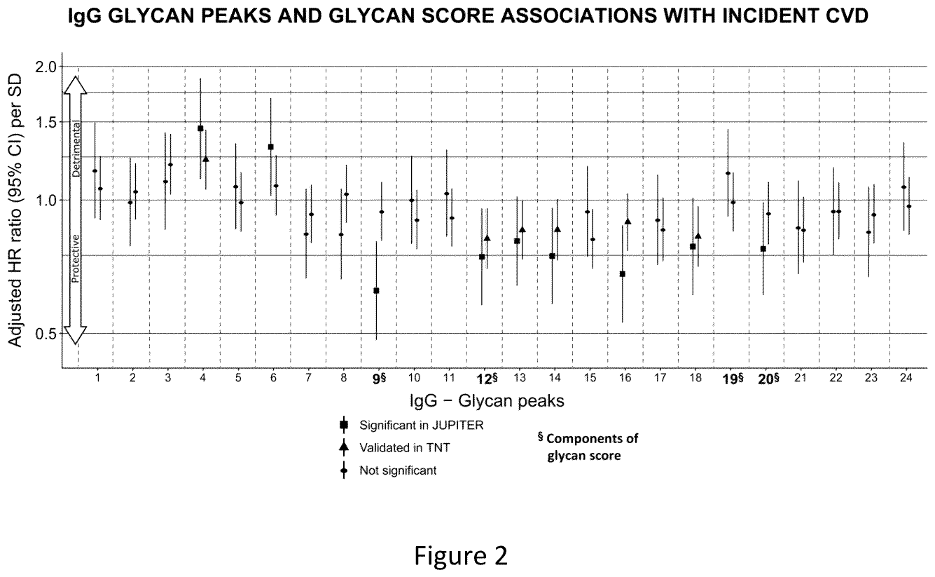

FIG. 2 displays HRs and 95% CI for the association of CVD events with each IgG-GP, adjusted for model 2 covariates. Nine individual GPs were associated with CVD in JUPITER (first stage FDR<0.2), which were carried over for validation in TNT. Significant results (second stage FDR<0.2) were detected for IgG-GP4, IgG-GP12, IgG-GP13, IgG-GP14, IgG-GP16, and IgG-GP18, and are highlighted in FIG. 2.

All analyses were corrected for multiple testing using overall two-stage FDR<0.05. Sensitivity analysis in participants with 12 months of follow-up measurements showed results consistent with baseline analysis in both cohorts.

Associations of Derived IgG N-Glycosylation Traits with CVD

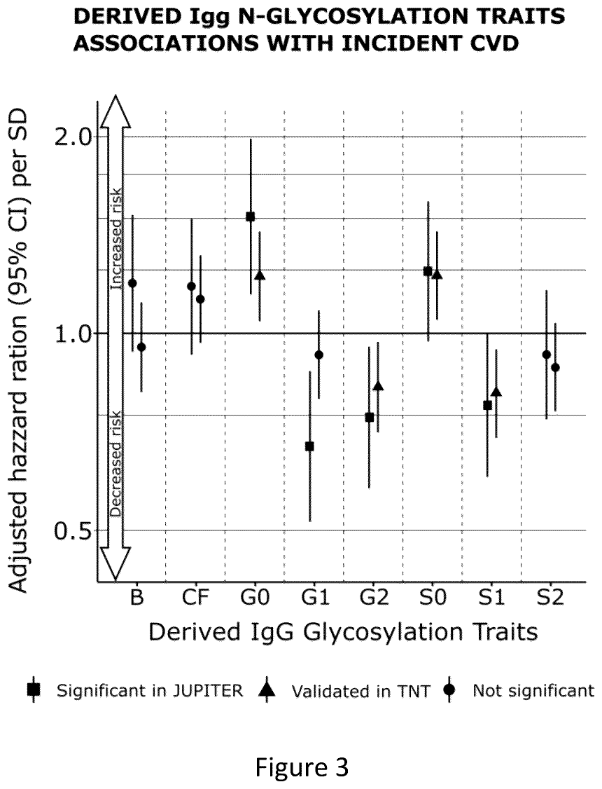

We examined the association of eight derived IgG N-glycan traits with CVD risk. Derived traits were calculated as percentages of the sums of glycans with specific common structural features, see literature reference 7. FIG. 3 displays HRs and 95% CIs for the relationship between each derived IgG N-glycan trait and CVD adjusted for model 2. The results were very consistent with the observed associations for individual IgG N-Glycans: agalactosylation and asialylation traits were FDR-adjusted and positively associated with CVD risk, while digalactosylation, and monosialylation were inversely associated.

Association of IgG Glycan Score with CVD Risk

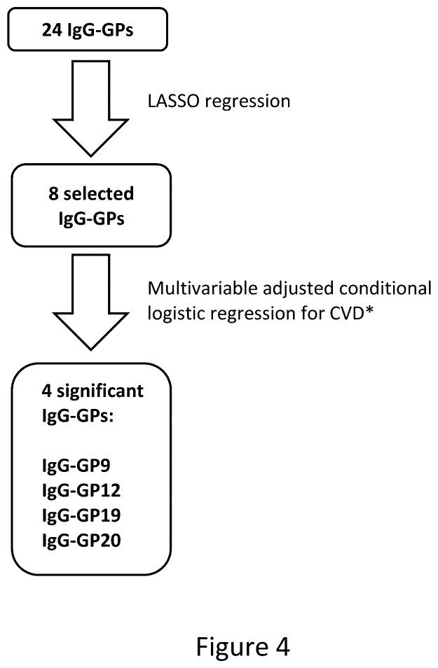

The IgG glycan score was derived in the discovery cohort, JUPITER. First, eight IgG-GPs with non-zero LASSO regression coefficients were selected. Second, these eight GPs were included in a conditional logistic regression model to test their association with CVD, adjusting for age, statin therapy, race, LDL-C, HDL-C, hypertension, and current smoking. This model yielded four IgG-GPs significantly associated with CVD (P<0.05): IgG-GP9, IgG-GP12, IgG-GP19, and IgG-GP20, see FIG. 4.

The formulas for the calculation of derived glycosylation traits: B (bisecting GlcNAc), CF (core fucosylation), G0 (agalactosylation), G1 (monogalactosylation), G2 (digalactosylation), S0 (asialylation), S1 (monosialylation), and S2 (disialylation) are given in Table 3.

TABLE 3
The calculation of derived IgG glycosylation traits that are subjected to the study
of potential association with incident cardiovascular disease (CVD), see FIG. 3.

| | Derived IgG glycosylation | |
|---|---|---|
| No. | trait | Formula |
| 1 | B: bisecting GlcNAc | (GP3 + GP6 + GP10 + GP11 + GP13 + GP15 + |
| | | GP19 + GP22 + GP24)/total GP · 100 |
| 2 | CF: core fucosylation | (GP1 + GP4 + GP6 + GP8 + GP9 + GP10 + GP11 + |
| | | GP14 + GP15 + GP16 + GP18 + GP19 + GP20 + |
| | | GP23 + GP24)/total GP · 100 |
| 3 | G0: agalactosylation | (GP1 + GP2 + GP3 + GP4 + GP6)/total GP · |
| | | 100 |
| 4 | G1: monogalactosylation | (GP7 + GP8 + GP9 + GP10 + GP11)/total GP · |
| | | 100 |
| 5 | G2: digalactosylation | (GP12 + GP13 + GP14 + GP15)/total GP . 100 |
| 6 | S0: asialylation | (GP1 + GP2 + GP3 + GP4 + GP6 + GP7 + GP8 + |
| | | GP9 + GP10 + GP11 + GP12 + GP13 + GP14 + |
| | | GP15)/total GP · 100 |
| 7 | S1: monosialylation | (GP16 + GP17 + GP18 + GP19 + GP20)/total |
| | | GP · 100 |
| 8 | S2: disialylation | (GP21 + GP22 + GP23 + GP24)/total GP · 100 |

Then, a new multivariable-adjusted conditional logistic regression model including only the four significant GPs, GP9, GP12, GP19, and GP20, plus age and sex was fitted to generate the S-coefficients used as weights for IgG glycan score for CVD, called cardiovascular disease risk (CVDR) parameter calculated as:

$$CVDR = - 3.432 \cdot GP 9 - 0.509 \cdot GP 12 + 0.793 \cdot GP 19 - 0.835 \cdot GP 20 \qquad ( Equation 1 )$$

In JUPITER, the IgG glycan score was associated with over two-fold higher CVD risk per SD increase in the score (HR=2.09, 95% CI=1.55-2.84 [adjusted for age, statin therapy, race, LDL-C, HDL-C, smoking, and hypertension], see Table 4:

TABLE 4
Associations of IgG glycan score and its four components with CVD in
JUPITER and validation in TNT.

| | JUPITER | | | TNT | | |
|---|---|---|---|---|---|---|
| | Model 1 | Model 2 | Model 3 | Model 1 | Model 2 | Model 3 |
| | HR (958 | HR (95% | HR (958 | HR (95% | HR (958 | HR (95% |
| IgG-GP | CI); p-value | CI); p-value | CI); p-value | CI); p-value | CI); p-value | CI); p-value |
| GP9 | 0.55 | 0.57 | 0.59 | 0.81 | 0.8 | 0.8 |
| | (0.42-0.73); | (0.43-0.76); | (0.44-0.79); | (0.69-0.95); | (0.68-0.94); | (0.67-0.94); |
| | 2.4e⁻⁰⁵ | 1.2e⁻⁰⁴ | 3.6e⁻⁰⁴ | 0.01 | 0.008 | 0.009 |
| GP12 | 0.73 | 0.71 | 0.70 | 0.88 | 0.88 | 0.89 |
| | (0.56-0.94); | (0.54-0.93); | (0.53-0.92); | (0.75-1.03); | (0.74-1.03); | (0.75-1.05); |
| | 0.017 | 0.11 | 0.010 | 0.108 | 0.113 | 0.167 |
| GP19 | 1.26 | 1.21 | 1.25 | 0.93 | 0.94 | 0.93 |
| | (0.98-1.62); | (0.93-1.57); | (0.95-1.64); | (0.79-1.09); | (0.79-1.11); | (0.78-1.11); |
| | 0.075 | 0.149 | 0.111 | 0.372 | 0.456 | 0.441 |
| GP20 | 0.75 | 0.74 | 0.76 | 1 | 1 | 1.05 |
| | (0.58-0.98); | (0.56-0.98); | (0.57-1.01); | (0.86-1.16); | (0.86-1.17); | (0.89-1.24); |
| | 0.036 | 0.036 | 0.062 | 0.989 | 0.985 | 0.548 |
| IgG | 2.16 | 2.11 | 2.09 | 1.22 | 1.22 | 1.23 |
| glycan | (1.60-2.91); | (1.55-2.87); | (1.53-2.87); | (1.04-1.42); | (1.04-1.43); | (1.05-1.45); |
| score | 4.5e⁻⁰⁷ | 1.8e⁻⁰⁶ | 4.3e⁻⁰⁶ | 0.013 | 0.014 | 0.012 |
| (Equation | | | | | | |
| 1) | | | | | | |

HRs (95% Cis) for single IgG-GPs are from multivariable-adjusted models. The IgG glycan score for CVD risk is the linear combination of the selected N-glycans, weighted by the regression coefficient from the mutually adjusted conditional logistic regression model and centered to mean = 0 and scaled to SD = 1. In JUPITER, model 1 was adjusted for age and statin treatment, model 2 was additionally adjusted for race, LDL-C, HDL-C, smoking, and prevalent hypertension, and model 3 was model 2 plus BMI and hs-CRP, Sex was a matching criterion for case-control selection. In TNT, model 1 was adjusted for age and sex, model 2 was additionally adjusted for race, LDL-C, HDL-C, hypertension, current smoking, and diabetes, and model 3 was model 2 plus BMI and hs-CRP. Treatment and disease risk scores were matching criteria for case-control selection.

Using a similar model, but additionally adjusting for sex and diabetes, the score was significantly validated in TNT (HR=1.202; 95% CI=1.027-1.41; p=0.022).

The AUC in JUPITER for a model with IgG glycan score plus age, statin therapy, race, LDL-C, HDL-C, smoking, and hypertension was 0.72 (95% CI=0.68-0.75) vs. 0.69 for the nested model without the score (95% CI=0.68-0.72). The P-value for the likelihood ratio test (P_{LRT}) was 2.3e⁻⁶. Similar models but adding sex and diabetes were used for replication in TNT, and the AUC was 0.71 (95% CI=0.70-0.83) vs. 0.64 (95?CI=0.63-0.70) for models with and without the score, respectively (P_{LRT}=0.02).

Interactions with Sex or Age

Considering IgG-GPs individual associations with CVD in JUPITER, we detected nominal significant effect modification by sex for IgG-GP20 in model 2 (for age, statin therapy, race, LDL-C, HDL-C, smoking, prevalent hypertension; P=0.047) and model 3 (model 2 plus BMI and hs-CRP; 0.015). Subgroup analysis by sex showed decreased risk for women (HR_{mode12}=0.43, 95% CI=0.22-0.85; HR_{mode13}=0.24, 95% CI=0.09-0.68) and non-significant association in men (HR_{mode12}=0.86, 95% CI=0.66-1.1; HR_{mode13}=0.91, 95% CI=0.69-1.2). No significant effect modification by age (above and below the median) was detected for the association between individual IgG-GPs and CVD risk (P>0.05). As for derived IgG N-glycosylation traits and IgG glycan score, no significant effect modification either by sex or age was detected (P>0.05).

Spearman Correlations of IgG Glycan Score, IgG-GPs, and Derived IgG N-Glycosylation Traits with Clinical Biomarkers

Significant results and consistency in terms of the sign were observed across both sub-studies for many biomarkers. Focusing on CVD-associated GPs and IgG glycan components, we highlight the IgG-GP4 positive correlation with hs-CRP and negative correlation with HDL-C, while IgG-GP14 positively correlated with HDL-C. One glycan score component, IgG-GP20, positively correlated with LDL-C, HDL-C, and total-C.

The present findings show robust and reproducible associations of IgG N-glycosylation profiles with the risk of future clinical CVD events. We detected an IgG N-glycan biosignature related to galactosylation and sialylation that may play an important role in CVD pathogenesis. Furthermore, we derived a weighted IgG glycan score that was also significantly and positively associated with CVD risk and which significantly improved model prediction performance. These associations were independent of confounders and replicated in a different population with distinct clinical characteristics.

Galactosylation of IgG acts as a modulator of its inflammatory activity and represents an interface between physiological and pathological processes. Moreover, galactosylation is also essential for subsequent sialic acid additions by glycosyltransferases, see literature reference 6. Our results suggest a predominant protective effect of IgG galactosylation and sialylation that potentially have a pivotal role in CVD prevention.

Strengths of this study include CVD associations with IgG glycan profile and IgG glycan score examined in two independent populations with distinct clinical characteristics. JUPITER was a CVD primary prevention study on individuals with high levels of hs-CRP and low LDL-C, whereas TNT was a secondary prevention coronary artery disease population. Furthermore, we highlight that the biomarker discovery associated with CVD in the validation cohort was independent of the covariates included in the models, and an extensive set of potential residual risk factors were incorporated in the disease risk score, used as one of the case-control matching criteria, see also literature reference 17. Underrepresentation of ethnicities other than white is a limitation of this study, and IgG glycobiology and its relationship with cardiometabolic risk in other ethnicities are warranted. Additional studies on CVD incidence-associated IgG N-glycans are needed to establish the generalizability of our results.

In summary, we showed that baseline IgG N-glycosylation profiles associate with the risk of future CVD, whether they are considered as individual IgG N-glycans, summed (derived traits), or weighted (glycan score) levels. Our results indicate that an IgG N-glycan biosignature related to galactosylation and sialylation may play an important role in CVD pathogenesis by regulating the pro- or anti-inflammatory responses of IgG. Therefore, IgG N-glycans and particularly those that correspond to glycan peaks GP9, GP12, GP19, and GP20 are of special value for the diagnostic prediction of future CVD onset in the subject being examined.

### Example 3. The Typical Procedure for Performing the CVDR Assessment Method According to the Present Disclosure

The typical procedure for performing the CVDR assessment of the subject being examined is as follows:
(i) isolation of plasma from one or more blood samples that have been collected from the subject under examination,
(ii) isolation of IgG from blood plasma,
(iii) the release of N-glycans from IgG,
(iv) fluorescent derivatization with 2-aminobenzamide (2AB),
(v) quantitative analysis of thus derivatized glycans by ultraperformance liquid chromatography (UPLC), wherein the numerical values of relative area under the respective glycan peaks GP1-GP24 within the corresponding UPLC chromatogram are obtained as the numerical values {GP1, GP2, . . . , GP24}, where the said glycan peaks GP1-GP24, and
(vi) the numerical results of relative areas under the selected glycan peaks GP9, GP12, GP19, and GP20, that correspond to values {GP9, GP12, GP19, GP20}calculated in the previous step, are now included in the numerical model for cardiovascular diseases risk (CVDR) as follows:

$$CVDR = - 3.432 \cdot GP 9 - 0.509 \cdot GP 12 + 0.793 \cdot GP 19 - 0.835 \cdot GP 20$$

where said CVDR value gives a qualitative assessment of cardiovascular disease (CVD) onset in the future for the examined subject as follows:
(a) qualitatively, if the CVDR value is greater than the mean CVDR value of the population, the subject has an increased risk of cardiovascular events in the future compared to the reference population, and
(b) quantitatively, the increase of CVDR value by 1 unit represents an increased risk of 2.6 fold,
where the mean CVDR value of the population depends on the characteristics of a given population regarding the territorial area where the test would be applied and would have to be determined on a representative sample of a population.

The detailed experimental procedures for steps (i)-(vi) are described in literature references 3 and 14, and in Example 1.

### INDUSTRIAL APPLICABILITY

The present disclosure discloses a diagnostic process for determining whether an examined subject has a future risk of cardiovascular disease (CVD) development from one or more blood samples. In this manner, the industrial applicability of the present disclosure is obvious.

### ABBREVIATIONS

The nomenclature of IgG N-glycans, e.g., FA1, A2, A2B, etc., is derived according to the rules of the Oxford nomenclature. The meaning of the abbreviations used is as follows:
2AB=2-aminobenzamide;
APTS=8-aminopyrene-1,3,6-trisulfonic acid, trisodium salt;
BMI=body mass index;
CI=confidence interval;
CVDR=cardiovascular disease risk;
DMSO=dimethyl sulfoxide, a solvent;
GP1 . . . GP24=peaks of IgG glycans in UPLC analytical method according to the present disclosure;
GU=glucose units; represent the retention time calibrated to the retention times of glucose oligomers labeled with 2-aminobenzamide; this reduces the variation in retention times of the system between separate runs;
HDL-C=high-density lipoprotein cholesterol;
HILIC=hydrophilic interaction liquid chromatography;
hs-CRP=high-sensitivity C-reactive protein;
IgG=immunoglobulin G;
LDL-C=low-density lipoprotein cholesterol;
2PB=2-picoline borane;
PBS=phosphate-buffered saline, a buffer solution;
PNGase F=enzyme peptide-N4-(N-acetyl-beta-glucosaminyl)asparagine amidase F;
PR=procainamide;
RF=2,5-dioxopyrrolidine-1-yl-[2N-(2-(N′,N′-diethylamino)ethyl) carbamoyl]-quinoline-6-yl-carbamate;
r.t.=room temperature;
RT=retention time (t4); retention time of the respective glycan peak in e.g., UPLC chromatogram;
Total-C=total cholesterol;
UPLC=ultra-high performance liquid chromatography.

1. A method for prediction of future cardiovascular disease (CVD) in a human subject comprises performing an analysis process of N-glycans I, bound to immunoglobulin G <IgG>, 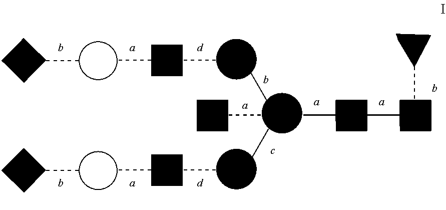
where various symbols in I denote monomeric sugar units as below:
▪ N-acetylglucosamine
▾ fucose
● C mannose
∘ galactose
♦ sialic acid
where letters a-d in I determine types of glycoside bond of said N-glycans I as below:
$$\begin{matrix}a = β 〈 1 - 4 〉 \\ b = α 〈 1 - 6 〉 \\ c = α 〈 1 - 3 〉 \\ d = β 〈 1 - 2 〉\end{matrix}$$
where the analysis process comprises following steps:
a) isolation of plasma from one or more blood samples that have been collected from the human subject under examination,
b) isolation of IgG from blood plasma of the blood samples,
c) release of said glycans from IgG,
d) fluorescent derivatization with 2-aminobenzamide <2AB> and a reducing agent for reductive amination, optionally using a complex of picoline borane <BH₃·NC₅H₄-2-CH₃> or sodium cyanoborohydride <NaBH₃CN>: 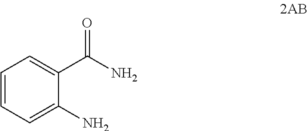
e) quantitative analysis of thus derivatized glycans by ultraperformance liquid chromatography <UPLC>, wherein numerical values of relative areas under respective glycan peaks GP 9, GP 12, GP 19, and GP 20 within the corresponding UPLC chromatogram are obtained as the numerical values {GP9, GP12,GP 19, GP20}, wherein the said glycan peaks GP 9, GP 12, and GP 19 belong to structures given in the table below:

| Glycan peak composition | Code | Structure |
|---|---|---|
| F(6)A2[3]G1 <100%> | GP9 | 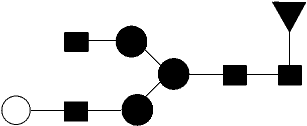 |
| A2G2 <91%> F(6)A2[3]BG1 <9%> | GP12 | 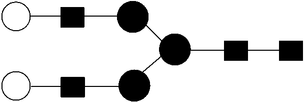 |
| | | 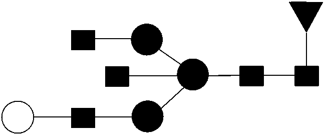 |
| F(6)A2BG2S1 <100%> | GP19 | 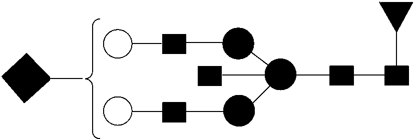 |

wherein G20 represents a peak at a retention time calibrated to retention times of glucose oligomers labeled with 2-aminobenzamide,
f) wherein numerical results of relative areas under the selected glycan peaks GP9, GP12, GP19, and GP20, that correspond to values {GP9, GP12, GP19, GP20}calculated in step e, are now included in a CVDR model for cardiovascular diseases risk <CVDR>, which is a function of the four glycan arguments:
CVDR=CVDR(GP9,GP12,GP19,GP20)
g) determination of the risk value expressed as the CVDR value.

2. The method according to claim 1, further comprising: obtaining the CVDR model via statistical data analysis performed after a prospective study that determines the variation of quantitative IgG glycans {GP1, . . . , GP24}content in the blood plasma in:
normal subjects, versus,
subjects who experienced the following cardiovascular disease clinical manifestations: myocardial infarction, stroke, coronary revascularization, unstable angina requiring hospitalization, or death,
wherein significantly predictive IgG glycans are determined using regression models that are corrected for multiple confounders, including age, race, sex, statin therapy, low-density lipoprotein cholesterol, high-density lipoprotein cholesterol, smoking, and hypertension, and a glycan-based predictive model is constructed based on the determined IgG glycans construct.

3. The method according to claim 1, where the CVDR model is:
$$CVDR = - 3.432 \cdot GP 9 - 0.509 \cdot GP 12 + 0.793 \cdot GP 19 - 0.835 \cdot GP 20$$
and where the determination of the risk value expressed as the CVDR value comprises:
(i) if the CVDR value is greater than a mean CVDR value of a population, the subject has an increased risk of cardiovascular events in the future compared to the reference population, and/or
(ii) the increase of CVDR value by 1 unit represents an increased risk of 2.6 fold.

4. The method according to claim 1, where the glycans under the peaks GP9, GP12, GP19, and GP20 are determined by alternative quantitative analytical techniques selected from the group consisting of: MALDI-TOF mass spectrometry, liquid chromatography coupled with mass spectrometry <LC-MS>, or capillary electrophoresis <CE>, where the corresponding glycans are derivatized with a fluorescent derivatizing agent selected from the group comprising 2-aminobenzamide <2AB>, 8-aminopyrene-1,3,6-trisulfonic acid, trisodium salt <APTS>, procainamide <PR>, or 2,5-dioxopyrrolidine-1-yl-<2N-<2-<N′,N′-diethylamino>ethyl>carbamoyl>-quinoline-6-yl-carbamate <RF> having respective structures as below: 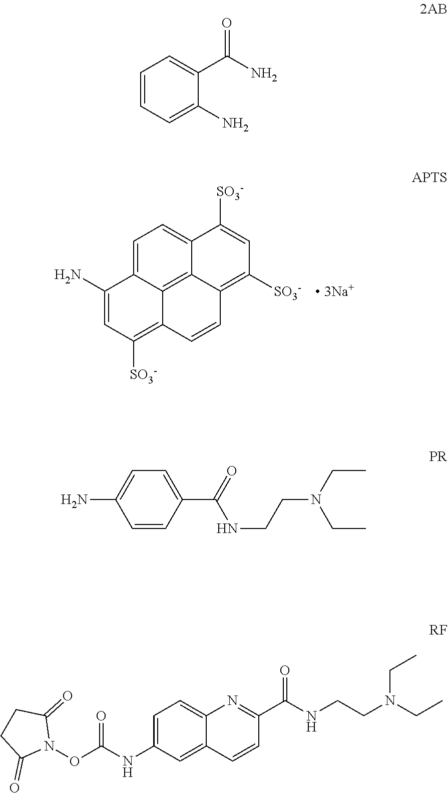
or by LC-MS analysis of the corresponding glycopeptides.

5. The method according to claim 2, where the glycans under the peaks GP9, GP12, GP19, and GP20 are determined by alternative quantitative analytical techniques selected from the group consisting of: MALDI-TOF mass spectrometry, liquid chromatography coupled with mass spectrometry <LC-MS>, or capillary electrophoresis <CE>, where the corresponding glycans are derivatized with a fluorescent derivatizing agent selected from the group comprising 2-aminobenzamide <2AB>, 8-aminopyrene-1,3,6-trisulfonic acid, trisodium salt <APTS>, procainamide <PR>, or 2,5-dioxopyrrolidine-1-yl-<2N-<2-<N′,N′-diethylamino>ethyl>carbamoyl>-quinoline-6-yl-carbamate <RF> having respective structures as below: 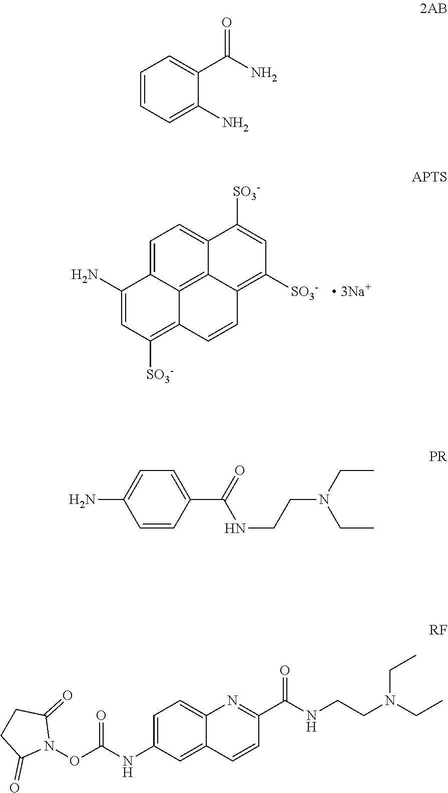
or by LC-MS analysis of the corresponding glycopeptides.

6. The method according to claim 3, where the glycans under the peaks GP9, GP12, GP19, and GP20 are determined by alternative quantitative analytical techniques selected from the group consisting of: MALDI-TOF mass spectrometry, liquid chromatography coupled with mass spectrometry <LC-MS>, or capillary electrophoresis <CE>, where the corresponding glycans are derivatized with a fluorescent derivatizing agent selected from the group comprising 2-aminobenzamide <2AB>, 8-aminopyrene-1,3,6-trisulfonic acid, trisodium salt <APTS>, procainamide <PR>, or 2,5-dioxopyrrolidine-1-yl-<2N-<2-<N′, N′-diethylamino>ethyl>carbamoyl>-quinoline-6-yl-carbamate <RF> having respective structures as below: 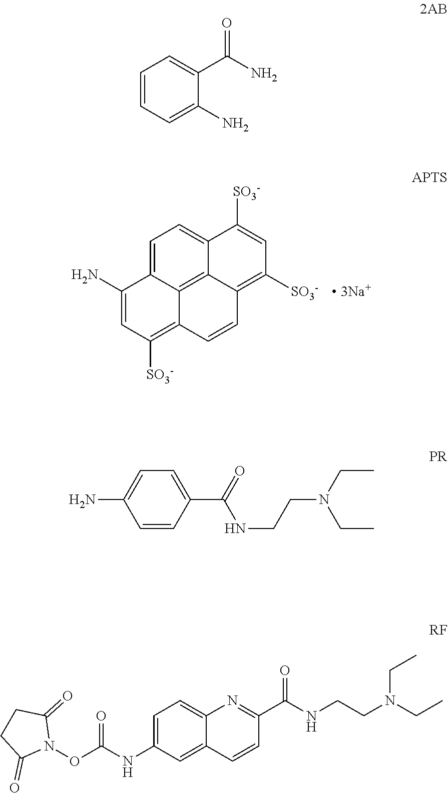
or by LC-MS analysis of the corresponding glycopeptides.

7. The method according to claim 1, further comprising: treating the human subject with a compound selected from the group consisting of acetylsalicylic acid, quercetin, resveratrol, N-acetyl-D-mannosamine, and combinations thereof if the human subject is determined to have an increased risk of cardiovascular events in the future.
